(19) United States
(12) Patent Application Publication
(10) Pub. No.: US 2024/0077710 A1
(43) Pub. Date: Mar. 7, 2024

(54) SURGICAL MICROSCOPE SYSTEM

(71) Applicant: Sony Olympus Medical Solutions Inc., Tokyo (JP)

(72) Inventors: Yasuhiro OKABE, Tokyo (JP); Masataka KADO, Tokyo (JP)

(73) Assignee: Sony Olympus Medical Solutions Inc., Tokyo (JP)

(21) Appl. No.: 18/261,840

(22) PCT Filed: Dec. 6, 2021
(86) PCT No.: PCT/JP2021/044792

§ 371 Date: Jul. 18, 2023

(30) Foreign Application Priority Data
Feb. 5, 2021 (JP) 2021-017761

(51) Int. Cl. G02B 21/00; G02B 21/06

(52) U.S. Cl. CPC G02B 21/0012; G02B 21/06; A61B 90/50

(57) ABSTRACT

A surgical microscope system of the present disclosure includes an imaging unit including an observation optical system configured using a plurality of optical members including an objective lens and an imaging element that captures a subject image formed by the observation optical system, and an illumination unit including a light source unit that emits light and an illumination optical system that guides the light emitted by the light source unit. The illumination optical system forms an illumination optical path independent of an observation optical path formed by the observation optical system. The illumination optical path intersects with the observation optical path on a subject side with respect to the objective lens.

### FIELD

The present disclosure relates to a surgical microscope system.

### BACKGROUND

Conventionally, as an observation system for observing a minute site in an eye or the like of a patient, which is an object to be observed, a known optical microscope system includes a support unit having a plurality of arms, and a microscope unit provided at a distal end of the support unit and having an enlargement optical system for enlarging the minute site and an imaging element. When performing an operation using this microscope system, an operator (user) such as a doctor moves the microscope unit and places the microscope unit at a desired position, and performs the operation while observing an image captured by the microscope unit.

In a cataract surgery, prevention of uncollected opacified lens in the step of suctioning a crushed lens greatly affects improvement in visual acuity after the surgery. In the cataract surgery using the microscope system, opacity can be observed by red reflection in which light entering the eye and reflected from retina is observed.

In the surgical microscope system, perfect coaxial illumination that separates illumination light and observation light using a beam splitter is used (Patent Literature 1). However, in the perfect coaxial illumination, there have been cases where the illumination light reflected inside the beam splitter creates glare and causes a double observation image, or stray light of an illumination optical system enters an image sensor or an eyepiece via the beam splitter and causes flare. As a countermeasure, a technique is known in which the illumination light is irradiated via an objective lens without providing the beam splitter (Patent Literature 2).

### CITATION LIST

### Patent Literature

Patent Literature 1: WO 2017/065018 A
Patent Literature 2: JP 2004-139002 A

### SUMMARY

### Technical Problem

However, in a configuration of Patent Literature 2, illumination light reflected in an observation optical system is flared and may result in an unclear observation image.

The present disclosure has been made in view of the above, and it is therefore an object of the present invention to provide a surgical microscope system capable of suppressing flare caused by reflection of the illumination light inside the observation optical system.

### Solution to Problem

To solve the above-described problem and achieve the object, a surgical microscope system according to the present disclosure includes: an imaging unit including an observation optical system configured using a plurality of optical members including an objective lens, and an imaging element configured to capture an image of a subject formed by the observation optical system; and an illumination unit including a light source unit configured to emit light, and an illumination optical system configured to guide the light emitted by the light source unit, wherein the illumination optical system is configured to form an illumination optical path that is independent of an observation optical path formed by the observation optical system, the illumination optical path intersecting with the observation optical path on a side of the subject with respect to the objective lens.

### Advantageous Effects of Invention

According to the present invention, it is possible to suppress flare caused by reflection of illumination light in an observation optical system.

### BRIEF DESCRIPTION OF DRAWINGS

FIG. 1 is a diagram illustrating a configuration of a surgical microscope system according to a first embodiment of the present disclosure.

FIG. 2 is a block diagram illustrating the configuration of the surgical microscope system according to the first embodiment of the present disclosure.

FIG. 3 is a diagram illustrating a configuration of a microscope unit of the surgical microscope system according to the first embodiment of the present disclosure.

FIG. 4 is a diagram illustrating the configuration of the microscope unit viewed from a direction of an arrow A in FIG. 3.

FIG. 5 is a diagram illustrating an angle of view and an illumination position of the microscope unit of the surgical microscope system according to the first embodiment of the present disclosure.

FIG. 6 is a diagram illustrating a configuration of the microscope unit of the surgical microscope system according to a first modification of the first embodiment of the present disclosure.

FIG. 7 is a diagram (part 1) illustrating mirror arrangement in the microscope unit of the surgical microscope system according to a second modification of the first embodiment of the present disclosure.

FIG. 8 is a diagram (part 2) illustrating the mirror arrangement in the microscope unit of the surgical microscope system according to the second modification of the first embodiment of the present disclosure.

FIG. 9 is a block diagram illustrating a configuration of the surgical microscope system according to a third modification of the first embodiment of the present disclosure.

FIG. 10 is a diagram illustrating a configuration of the microscope unit of the surgical microscope system according to the third modification of the first embodiment of the present disclosure.

FIG. 11 is a diagram illustrating an angle of view and an illumination position of the microscope unit of the surgical microscope system according to the third modification of the first embodiment of the present disclosure.

FIG. 12 is a diagram illustrating a configuration of the microscope unit of the surgical microscope system according to a fourth modification of the first embodiment of the present disclosure.

FIG. 13 is a diagram illustrating an angle of view and an illumination position of the microscope unit of the surgical microscope system according to the fourth modification of the first embodiment of the present disclosure.

FIG. 14 is a diagram illustrating a configuration of the microscope unit of the surgical microscope system according to a fifth modification of the first embodiment of the present disclosure.

FIG. 15 is a diagram illustrating the configuration of the microscope unit viewed from a direction of an arrow D in FIG. 14.

FIG. 16 is a diagram illustrating the configuration of the microscope unit viewed from a direction of an arrow E in FIG. 14.

FIG. 17 is a diagram illustrating a configuration of the microscope unit of the surgical microscope system according to a sixth modification of the first embodiment of the present disclosure.

FIG. 18 is a diagram illustrating the configuration of the microscope unit viewed from a direction of an arrow F in FIG. 17.

FIG. 19 is a diagram illustrating the configuration of the microscope unit viewed from a direction of an arrow G in FIG. 17.

FIG. 20 is a diagram illustrating a configuration of a microscope unit of a surgical microscope system according to a second embodiment of the present disclosure.

FIG. 21 is a diagram illustrating a configuration of a microscope unit of a surgical microscope system according to a third embodiment of the present disclosure.

FIG. 22 is a diagram illustrating a configuration of a surgical microscope system according to a fourth embodiment of the present disclosure.

### DESCRIPTION OF EMBODIMENTS

Hereinafter, modes for carrying out the present invention (hereinafter referred to as embodiments) will be described with reference to the accompanying drawings. Note that the drawings are merely schematic, and portions having different dimensional relationships and ratios may be included between the drawings.

### First Embodiment

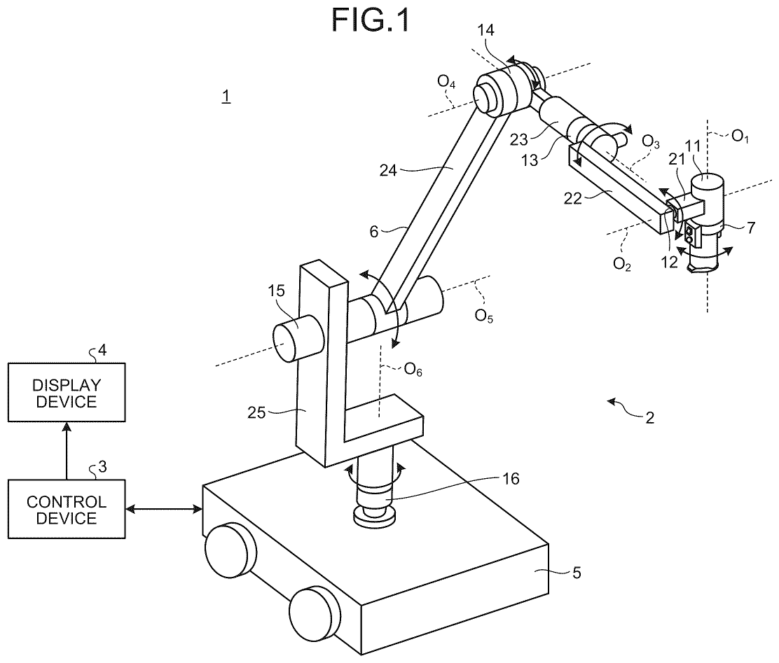

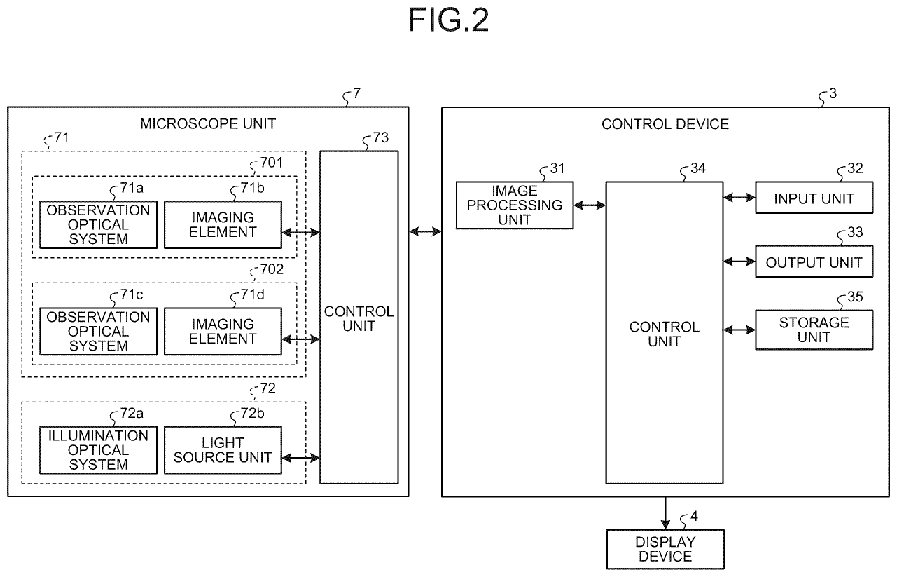

FIG. 1 is a diagram illustrating a configuration of a surgical microscope system according to a first embodiment. FIG. 2 is a block diagram illustrating the configuration of the surgical microscope system according to the first embodiment. A surgical microscope system 1 includes a microscope device 2 having a function as a microscope that enlarges and captures a microstructure of an object to be observed (subject), a control device 3 that integrally controls operation of the surgical microscope system 1, and a display device 4 that displays an image captured by the microscope device 2.

The control device 3 receives an imaging signal output from the microscope device 2 and performs predetermined signal processing on the imaging signal to generate image data for display. The control device 3 includes an image processing unit 31, an input unit 32, an output unit 33, a control unit 34, and a storage unit 35. Note that the control device 3 may be provided with a power supply unit (not illustrated) or the like that generates a power supply voltage for driving the microscope device 2 and the control device 3, supplies the power supply voltage to each unit of the control device 3, and supplies the power supply voltage to the microscope device 2 via a transmission cable.

The image processing unit 31 performs processing on the imaging signal output from a microscope unit 7 to generate a display image. The image processing unit 31 performs noise removal and signal processing such as A/D conversion, detection, interpolation, and color correction as necessary. The image signal generated by the image processing unit 31 is output to the display device 4 and displayed on the display device 4.

Furthermore, the image processing unit 31 may include an AF processing unit that outputs predetermined frame AF evaluation values based on the input imaging signal of the frame, and an AF calculation unit that performs an AF calculation process for selecting a frame, a focus lens position, or the like most suitable as a focus position in the frame AF evaluation values from the AF processing unit.

The input unit 32 is realized by using a user interface such as a keyboard, a mouse, or a touch panel, and receives inputs of various types of information.

The output unit 33 is realized by using a speaker, a printer, a display, or the like, and outputs various types of information.

The control unit 34 performs drive control of each part including the microscope device 2 and the control device 3, and input/output control of information with respect to each component. The control unit 34 generates a control signal with reference to communication information data (e.g., communication format information) recorded in the storage unit 35, and transmits the control signal generated to the microscope device 2.

Note that the control unit 34 generates a synchronization signal and a clock for the microscope unit 7 and the control device 3. The synchronization signal (e.g., synchronization signal instructing imaging timing.) and the clock (e.g., clock for serial communication) to the microscope unit 7 are sent to the microscope unit 7 through a line not illustrated, and the microscope unit 7 is driven based on these synchronization signal and clock.

The storage unit 35 is realized by using a semiconductor memory such as a flash memory or a dynamic random access memory (DRAM), and stores the communication information data (e.g., communication format information) and the like. Note that various programs executed by the control unit 34 may be stored in the storage unit 35.

The image processing unit 31 and the control unit 34 described above are realized using a general-purpose processor such as a central processing unit (CPU) having an internal memory (not illustrated) in which a program is recorded or a dedicated processor such as various arithmetic circuits that execute specific functions such as an application specific integrated circuit (ASIC). Alternatively, a field programmable gate array (FPGA, not illustrated), which is one type of programmable integrated circuit, may be used. When the FPGA is used, a memory for storing configuration data may be provided, and the FPGA as a programmable integrated circuit may be configured by the configuration data read from the memory.

The display device 4 receives the image data generated by the control device 3 from the control device 3 and displays an image corresponding to the image data. This display device 4 includes a display panel made of liquid crystal or organic electro luminescence (EL). Note that, in addition to the display device 4, an output device that outputs information using a speaker, a printer, or the like may be provided.

The microscope device 2 includes a base unit 5 movable on a floor surface, a support unit 6 supported by the base unit 5, and a columnar microscope unit 7 provided at a distal end of the support unit 6 to enlarge and capture a minute site of the object to be observed. Note that the control device 3 may be installed inside the base unit 5 and integrated with the microscope device 2.

In the microscope device 2, for example, a cable group including a transmission cable such as a signal line for performing signal transmission between the control device 3 and the microscope unit 7 is arranged from the base unit 5 to the microscope unit 7.

The support unit 6 includes, for example, a first joint 11, a first arm 21, a second joint 12, a second arm 22, a third joint 13, a third arm 23, a fourth joint 14, a fourth arm 24, a fifth joint 15, a fifth arm 25, and a sixth joint 16.

The support unit 6 includes four sets each including two arms and a joint that rotatably connects one (distal end side) of the two arms to the other (proximal end side). Specifically, these four sets are (first arm 21, second joint 12, and second arm 22), (second arm 22, third joint 13, and third arm 23), (third arm 23, fourth joint 14, and fourth arm 24), and (fourth arm 24, fifth joint 15, and fifth arm 25).

The first joint 11 rotatably holds the microscope unit 7 on the distal end side, and the proximal end side of the first joint 11 is held by the first arm 21 in a state of being fixed to the distal end of the first arm 21. The first joint 11 is cylindrical and holds the microscope unit 7 so as to be rotatable around a first axis O₁ that is a central axis in a height direction. The first arm 21 has a shape extending from a side surface of the first joint 11 in a direction orthogonal to the first axis O₁.

The second joint 12 rotatably holds the first arm 21 on the distal end side, and the proximal end side of the second joint 12 is held by the second arm 22 in a state of being fixed to a distal end portion of the second arm 22. The second joint 12 is cylindrical, and holds the first arm 21 so as to be rotatable around a second axis O₂ that is a central axis in the height direction and orthogonal to the first axis O₁. The second arm 22 has a substantially L shape, and is connected to the second joint 12 at an end of a vertical portion of the L shape.

The third joint 13 rotatably holds a horizontal portion of the L shape of the second arm 22 on the distal end side, and the proximal end side of the third joint 13 is held by the third arm 23 in a state of being fixed to a distal end portion of the third arm 23. The third joint 13 is cylindrical, and holds the second arm 22 so as to be rotatable around a third axis O₃ that is a central axis in the height direction, orthogonal to the second axis O₂, and parallel to a direction in which the second arm 22 extends. The third arm 23 has a cylindrical shape on the distal end side, and a through hole is created on the proximal end side in a direction orthogonal to the height direction of the cylindrical shape on the distal end side. The third arm 23 is rotatably held by the fourth joint 14 via the hole.

The fourth joint 14 rotatably holds the third arm 23 on the distal end side, and the proximal end side of the fourth joint 14 is held by the fourth arm 24 in a state of being fixed to the fourth arm 24. The fourth joint 14 is cylindrical, and holds the third arm 23 so as to be rotatable around a fourth axis O₄ that is the central axis in the height direction and orthogonal to the third axis 03.

The fifth joint 15 rotatably holds the fourth arm 24 on the distal end side, and the proximal end side of the fifth joint 15 is fixedly attached to the fifth arm 25. The fifth joint 15 is cylindrical, and rotatably holds the fourth arm 24 around a fifth axis O₅ that is the central axis in the height direction and parallel to the fourth axis O₄. The fifth arm 25 has an L-shaped portion and a rod-shaped portion extending downward from a horizontal portion of the L shape. The proximal end side of the fifth joint 15 is attached to an end of a vertical portion of the L shape of the fifth arm 25.

The sixth joint 16 rotatably holds the fifth arm on the distal end side, and the proximal end side of the sixth joint 16 is fixedly attached to an upper surface of the base unit 5. The sixth joint 16 is cylindrical, and rotatably holds the fifth arm 25 around a sixth axis O₆ that is the central axis in the height direction and orthogonal to the fifth axis O₅. The proximal end of the rod-shaped portion of the fifth arm 25 is attached to the distal end side of the sixth joint 16.

The support unit 6 as configured above realizes six-degree-of-freedom-movement, in total, of the microscope unit 7, that is, three degrees of freedom in translation and three degrees of freedom in rotation. Note that the support unit 6 is not limited to the above-described configuration as long as it supports the microscope unit 7. The number of arms and joints and an arm mechanism are not limited thereto.

The first joint 11 to the sixth joint 16 include electromagnetic brakes that prohibit the rotation of the microscope unit 7 and the first arm 21 to the fifth arm 25, respectively. Each of the electromagnetic brakes is released in a state where an arm control switch (described later) provided in the microscope unit 7 is pressed, so that the microscope unit 7 and the first arm 21 to the fifth arm 25 are allowed to rotate. Note that an air brake may be applied instead of the electromagnetic brake.

In addition to the electromagnetic brake described above, an encoder and an actuator may be mounted on each of the joints. For example, when the encoder is provided in the first joint 11, the encoder detects a rotation angle in the first axis O₁. The actuator includes, for example, an electric motor such as a servo motor, driven by control from the control device 3, and causes the joint to rotate for a predetermined angle. The rotation angle of the joint is set by the control device 3, for example, based on the rotation angle of each of the rotation axes (the first axis O₁ to the sixth axis O₆) as a value necessary for moving the microscope unit 7. As described above, the joints provided with an active driving system such as the actuator configure rotation shafts that actively rotate by controlling the drive of the actuator.

The microscope unit 7 is a cylindrical casing, and includes an imaging unit 71 that enlarges and captures an image of the object to be observed, two illumination units 72 that irradiate the object to be observed with illumination light, and a control unit 73 that controls the imaging unit 71 and the illumination units 72 under the control of the control device 3. The casing configuring the microscope unit 7 corresponds to a holding unit that holds the imaging unit 71 and the illumination units 72. In addition, the microscope unit 7 is provided with an arm control switch that receives an operation input to release the electromagnetic brake in the first joint 11 to the sixth joint 16 and permits the rotation of each joint, and a cross-type lever that can change magnification of the imaging unit and a focal length to the object to be observed. While the user presses the arm control switch, the electromagnetic brakes of the first joint 11 to the sixth joint 16 are released.

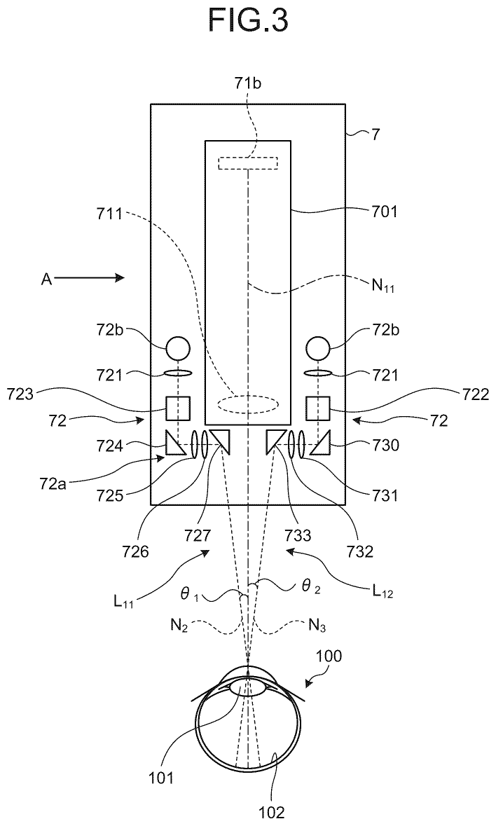

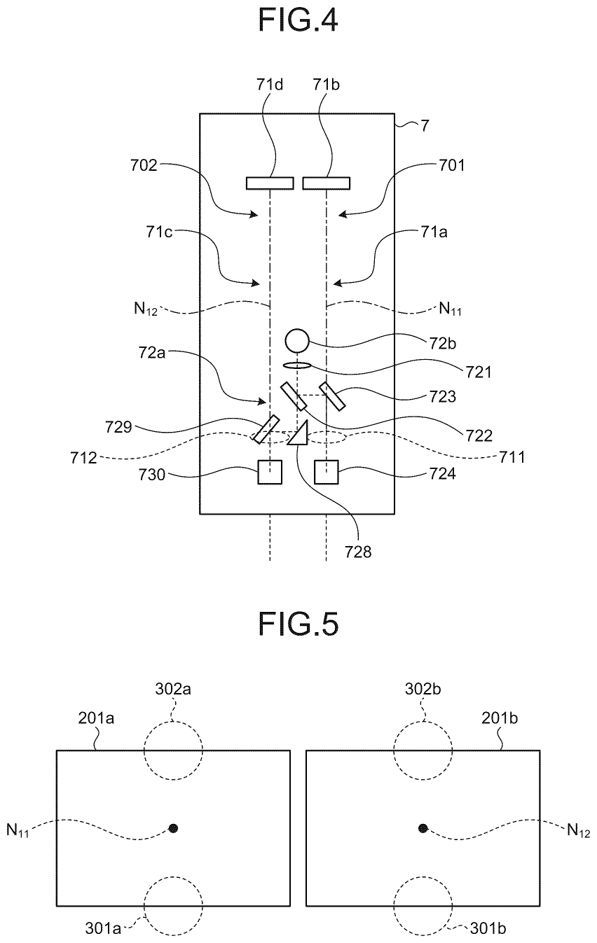

FIG. 3 is a diagram illustrating a configuration of the microscope unit of the surgical microscope system according to the first embodiment of the present disclosure. FIG. 4 is a diagram illustrating a configuration of the microscope unit viewed from a direction of an arrow A in FIG. 3. The imaging unit 71 captures an image of the subject by taking in observation light.

The imaging unit 71 is a two-eye imaging unit including a first imaging unit 701 and a second imaging unit 702. The first imaging unit 701 is configured by accommodating, in the casing, an observation optical system 71a that includes a plurality of lenses such as an objective lens 711 to form an image of observation light, and an imaging element 71b that takes in the observation light entering via the observation optical system 71a to generate an electric signal. The second imaging unit 702 is configured by accommodating, in the casing, an observation optical system 71c that includes a plurality of lenses such as an objective lens 712 to form an image of observation light, and an imaging element 71d that takes in the observation light entering via the observation optical system 71c to generate an electric signal. The imaging elements 71b and 71d receive and photoelectrically convert subject images formed by the observation optical systems 71a and 71c to generate electrical signals (imaging signals). The imaging elements 71b and 71d include a charge coupled device (CCD) image sensor or a complementary metal oxide semiconductor (CMOS) image sensor. The observation optical system 71a forms the subject image on an imaging surface of the imaging element 71b, and the observation optical system 71c forms the subject image on an imaging surface of the imaging element 71d. Note that an observation optical axis of an optical system formed by the first imaging unit 701 is an observation optical axis N₁₁, and an observation optical axis of an optical system formed by the second imaging unit 702 is an observation optical axis Ni₂. In each observation optical system, illustration of lenses other than the objective lens is omitted.

In the imaging unit 71, electric signals corresponding to two images having parallax are generated by the two imaging elements 71b and 71d. The image processing unit 31 generates a three-dimensional image based on the two electric signals. The operator wears, for example, stereoscopic glasses and observes the three-dimensional image.

The illumination unit 72 includes an illumination optical system 72a and a light source unit 72b.

The illumination optical system 72a guides light emitted from the light source unit 72b and emits the guided light to outside (object to be observed). The illumination optical system 72a includes a light source lens 721, a half mirror 722, a first mirror 723, a second mirror 724, relay lenses 725 and 726, a third mirror 727, a fourth mirror 728, a fifth mirror 729, a sixth mirror 730, relay lenses 731 and 732, and a seventh mirror 733. The half mirror 722 is, for example, an optical splitting unit that transmits half light with respect to a light amount and bends the remaining half light. Note that the half mirror 722 may be replaced with a beam splitter having a transmission/reflection ratio other than 1: 1.

In the illumination optical system 72a, light is split into two optical paths by the half mirror 722. In the illumination optical system 72a, the light emitted from the light source unit 72b is guided from the half mirror 722 to the vicinity of the observation optical axis N₁ of the observation optical system 71a via the relay lenses 725 and 726, and is guided by the third mirror 727 to travel toward the object to be observed. Specifically, in the illumination optical system 72a, the light emitted from the light source unit 72b is bent by the half mirror 722 and the first mirror 723 via the light source lens 721, and then bent by the second mirror 724. The light bent by the second mirror 724 is bent by the third mirror 727 via the relay lenses 725 and 726. The light bent by the third mirror 727 is emitted to the outside as the illumination light. Still more, the light emitted from the light source unit 72b and passing through the half mirror 722 via the light source lens 721 is bent by the fourth mirror 728 and the fifth mirror 729, and then bent by the sixth mirror 730. The light bent by the sixth mirror 730 is bent by the seventh mirror 733 via the relay lenses 731 and 732. The light bent by the seventh mirror 733 is emitted to the outside as the illumination light.

Here, the illumination optical system 72a is provided at a position deviated from the optical path of the observation light entering the imaging unit 71. In FIG. 3, the third mirror 727 and the seventh mirror 733 are provided at positions that do not disturb the light to be taken in by the objective lenses 711 and 712. The illumination optical system 72a forms independent optical paths L₁₁ and L₁₂ different from the observation optical system 71a. The optical paths L₁₁ and L₁₂ do not intersect or overlap with the optical paths from the objective lens 711 to the imaging element 71b and from the objective lens 712 to the imaging element 71d in the observation optical system 71a.

The light source unit 72b emits light including light in a wavelength band necessary for observation, such as white light, as the illumination light. For example, the white light includes light in all wavelength bands in a visible range.

Each of the two illumination units 72 includes the illumination optical system 72a and the light source unit 72b. The illumination units 72 are provided on opposite sides of the imaging unit 71. In other words, the illumination units 72 are arranged on the sides facing each other with the observation optical axes N₁₁ and N₁₂ interposed therebetween. The illumination units 72 emit the illumination light such that the illumination optical axes N₂ and N₃ intersect with the observation optical axes N₁₁ and N₁₂, respectively.

Angles θ₁ and θ₂ formed by the observation optical axes N₁₁ and N₁₂ of the imaging unit 71 and the optical axes of the illumination light (illumination optical axes N₂ and N₃) respectively emitted from the third mirror 727 and the seventh mirror 733 are 2.0 degrees or less, and more preferably 1.5 degrees or less. The angles θ₁ and θ₂ are preferably coaxial with the observation optical axis N₁ (close to zero degrees) in order to brightly illuminate a central portion of the object to be observed. For example, the angles θ₁ and θ₂ are set to about 1.5 degrees due to the arrangement of the objective lens 711 and the third mirror 727 in the microscope unit 7. The illumination light emitted from the third mirror 727 of the illumination unit 72 intersects with the observation optical axis N₁. For example, when the object to be observed is an eye 100 illustrated in FIG. 3, the illumination light intersects in the eye 100, for example, on a lens 101 or a retina 102.

FIG. 5 is a diagram illustrating an angle of view and an illumination position of the microscope unit of the surgical microscope system according to the first embodiment of the present disclosure. In one illumination unit 72 of the microscope unit 7, the third mirror 727 emits illumination light 301a from a position partially overlapping an edge end of an angle of view 201a, and the seventh mirror 733 emits illumination light 301b from a position partially overlapping an edge end of an angle of view 201b. In the other illumination unit 72, the third mirror 727 emits illumination light 302a from a position partially overlapping an edge end of the angle of view 201a, and the seventh mirror 733 emits illumination light 302b from a position partially overlapping an edge end of the angle of view 201b. Here, the illumination lights 301a and 302a are located on opposite sides of the observation optical axis N₁₁, and the illumination lights 301b and 302b are located on opposite sides of the observation optical axis N 12. The illumination lights 301a and 301b and the illumination lights 302a and 302b are located, for example, in the vertical direction of the eye that is the object to be observed.

The control unit 73 performs drive control of each component of the imaging unit 71 and the illumination unit 72, and input/output control of information with respect to each component. The control unit 73 is realized using a general-purpose processor such as a CPU having an internal memory (not illustrated) in which a program is recorded or a dedicated processor such as various arithmetic circuits that execute specific functions such as an ASIC. In addition, the FPGA, which is one type of programmable integrated circuit, may be used.

An outline of surgery performed using the surgical microscope system 1 having the above configuration will be described. When the operator who is the user performs an operation on an eye of a patient that is the object to be observed, the operator grips and moves the microscope unit 7 to a desired position in a state where the arm control switch of the microscope unit 7 is pressed while viewing the image displayed by the display device 4, determines the imaging visual field of the microscope unit 7, and then releases the finger from the arm control switch. As a result, the electromagnetic brake is applied to the first joint 11 to the sixth joint 16, and the imaging field of view of the microscope unit 7 will be fixed. Thereafter, the operator adjusts the magnification and the focal length to the object to be observed. Here, the illumination light is emitted from the microscope unit 7 via the illumination optical system 72a.

In the first embodiment described above, the illumination optical system 72a is configured independently of the observation optical systems 71a and 71c, and the illumination light is emitted to the outside of the microscope unit 7 via the illumination optical system 72a without passing through the objective lenses 711 and 712. According to the first embodiment, since the illumination light is emitted without passing through the observation optical system 71a, it is possible to suppress flare caused by reflection of the illumination light inside the observation optical system.

In the conventional perfect coaxial illumination, the light from the light source is reflected by the half mirror toward the object to be observed, and the light from the object to be observed that has passed through the half mirror is received. Therefore, when the half mirror reflects a half of the incident light and transmits a remaining half in the perfect coaxial illumination, an amount of light entering the imaging element will be about ¼ at the maximum with respect to an amount of light emitted from the light source. On the other hand, in the first embodiment, since the illumination optical system 72a that does not reduce the light amount by the half mirror is used, almost the entire light amount emitted from the light source is irradiated to the object to be observed, and the observation light enters the imaging element. According to the first embodiment, since observation brightness on a screen and the light amount entering the imaging element are in a proportional relationship, it is possible to perform brighter observation as the amount of light entering the imaging element increases. Here, a light amount La (constant) required for surgery, a light amount Lb entering the imaging element, and a light amount Lc received by the object to be observed achieve La=Lb, and the light amount entering the eye decreases as Lc decreases. By reducing the light amount entering the eye, a minimally invasive treatment can be performed. In the case of the perfect coaxial illumination, Lc=2×La is achieved, whereas in the case of the first embodiment, Lc=La is achieved. Therefore, the system according to the first embodiment can perform treatment twice less invasively as compared with the perfect coaxial illumination. In addition, according to the first embodiment, it is possible to observe the object to be observed brightly while suppressing the light amount from the light source as compared with the perfect coaxial illumination.

In addition, according to the first embodiment, since the illumination optical system 72a is independently of the observation optical system 71a, the illumination optical system 72a can be easily replaced. As a result, it is possible to easily change the illumination optical system, replace with a new lens, or the like.

In the first embodiment, the two illumination units 72 irradiate the object to be observed with the illumination light from different directions with respect to the observation optical axes N₁₁ and N₁₂. According to the first embodiment, since the object to be observed is irradiated with the illumination light from directions different from each other, high visibility of the object to be observed can be realized.

### First Modification of First Embodiment

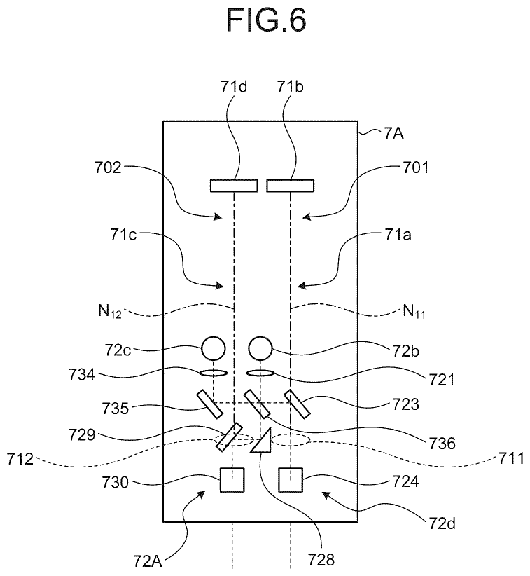

Next, a first modification of the first embodiment will be described with reference to FIG. 6. FIG. 6 is a diagram illustrating a configuration of the microscope unit of the surgical microscope system according to the first modification of the first embodiment of the present disclosure. The configuration of the surgical microscope system according to the first modification includes a microscope unit 7A instead of the microscope unit 7 of the surgical microscope system 1 according to the first embodiment described above. Since the configuration other than the microscope unit 7A is the same as that of the first embodiment, the description thereof will be omitted. Hereinafter, portions different from those of the first embodiment will be described.

The microscope unit 7A includes an illumination unit 72A instead of the illumination unit 72 of the microscope unit 7 described above. Since the configuration other than illumination unit 72A is the same as that of the first embodiment, the description thereof is omitted.

The illumination unit 72A includes an illumination optical system 72d and light source units 72b and 72c. Hereinafter, for the sake of explanation, the light source unit 72b is referred to as a first light source unit 72b, and the light source unit 72c is referred to as a second light source unit 72c. The first light source unit 72b and the second light source unit 72c are driven under the control of the control unit 73.

The second light source unit 72c is light in a wavelength band different from the wavelength band of the visible range. Specifically, the light emitted from the second light source unit 72c has a wavelength band different from a wavelength band of the light emitted from the first light source unit 72b, and is light in a wavelength band outside the visible range (e.g., near-infrared range) or a partial wavelength range (e.g., a part of a green wavelength band, a part of a blue wavelength band, or a wavelength band obtained by a combination thereof) of the wavelength band in the visible range, and is used for observation different from white light. In particular, in a cataract surgery, infrared light (near-infrared light) having a central wavelength around 850 nm is used.

The illumination optical system 72d splits the light emitted from the first light source unit 72b or the second light source unit 72c and emits the illumination light. The illumination optical system 72d includes the light source lens 721, a half mirror 736, the first mirror 723, the second mirror 724, the relay lenses 725 and 726, the third mirror 727, the fourth mirror 728, the fifth mirror 729, the sixth mirror 730, the relay lenses 731 and 732, the seventh mirror 733, a light source lens 734, and an eighth mirror 735. The half mirror 736 transmits, for example, half light with respect to a light amount and bends remaining half light.

In the illumination optical system 72d, similarly to the illumination optical system 72a, the half mirror 736 splits the light into two optical paths. Specifically, the light emitted from the first light source unit 72b passes through the light source lens 721, partially passes through the half mirror 736, and the remaining light is bent by the half mirror 736. On the other hand, the light emitted from the second light source unit 72c is bent by the eighth mirror 735 via the light source lens 734, a part of the bent light passes through the half mirror 736, and the remaining light is bent by the half mirror 736. The light path after the half mirror 736 is similar to that of the illumination optical system 72a.

In the first modification described above, the illumination optical system 72d independent of the observation optical systems 71a and 71c is configured, and the illumination light is emitted to the outside of the microscope unit 7A through the illumination optical system 72d without passing through the objective lenses 711 and 712. According to the first modification, since the illumination light is emitted without passing through the observation optical systems 71a and 71c, it is possible to suppress flare generated by reflection of the illumination light inside the observation optical system.

Further, in the first modification, the two illumination units 72A irradiate the object to be observed with the illumination light from different directions with respect to the observation optical axes N₁₁ and N₁₂. According to the first modification, since the object to be observed is irradiated with the illumination light from directions different from each other, high visibility of the object to be observed can be realized.

Furthermore, according to the first modification, it is possible to selectively switch the wavelength band of the light with which the object to be observed is irradiated by switching the driving of the light sources that emit the light in the wavelength bands different from each other.

### Second Modification of First Embodiment

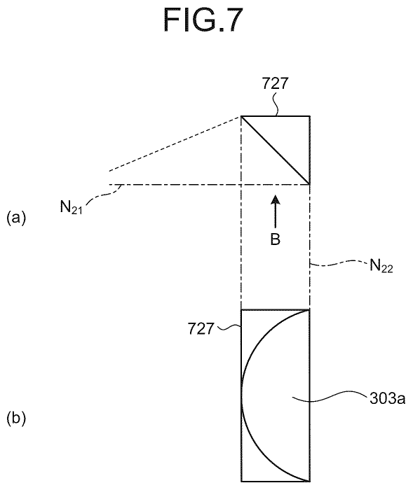

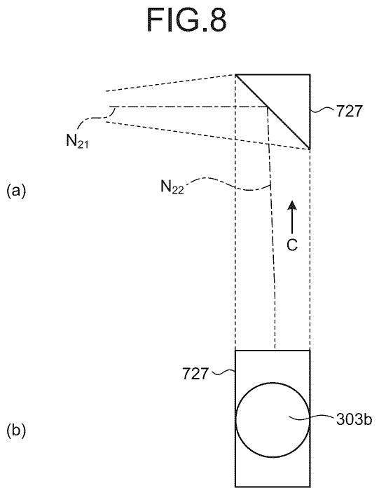

Next, a second modification of the first embodiment will be described with reference to FIGS. 7 and 8. FIGS. 7 and 8 are diagrams illustrating mirror arrangement in the microscope unit of the surgical microscope system according to the second modification of the first embodiment of the present disclosure. (b) of FIG. 7 is a diagram illustrating a cross section of the light viewed from a direction of an arrow B (illumination optical axis N₂₂) in (a) of FIG. 7. (b) of FIG. 8 is a diagram illustrating a cross section of the light viewed from a direction of an arrow C (illumination optical axis N₂₂) in (a) of FIG. 8. Since the configuration of the surgical microscope system according to the second modification is the same as the configuration of the surgical microscope system 1 of the first embodiment described above, the description thereof will be omitted. Hereinafter, portions different from those of the first embodiment will be described.

An arrangement of the third mirror 727 in the second modification differs from the above-described illumination optical system 72a. The third mirror 727 is disposed at a position reflecting a half of the light reflected by the second mirror 724. Specifically, one end side of the third mirror 727 reflects an edge end of the light, and the other end side reflects light near the center of the light (illumination optical axis N₂₁). As illustrated in FIG. 7, light 303a reflected by the third mirror 727 has a semicircular cross section.

On the other hand, in the first embodiment, for example, when the third mirror 727 is disposed at a position reflecting all the light reflected by the second mirror 724, illumination light 303b reflected by the third mirror 727 has a circular cross section (see FIG. 8).

In the second modification described above, similarly to the first embodiment, the illumination optical system 72a is configured independently of the observation optical systems 71a and 71c, and the illumination light is emitted to the outside of the microscope unit 7 via the illumination optical system 72a without passing through the objective lenses 711 and 712. According to the second modification, since the illumination light is emitted without passing through the observation optical systems 71a and 71c, it is possible to suppress flare generated by reflection of the illumination light inside the observation optical system.

In the second modification, since the third mirror 727 reflects the light from one end to the center (illumination optical axis) of light flux, the light amount at a center portion of the light emitted to the outside as the illumination light increases. According to the second modification, coaxiality between the observation optical axis and the illumination light (illumination optical axis) can be further improved, and more efficient illumination can be realized.

### Third Modification of First Embodiment

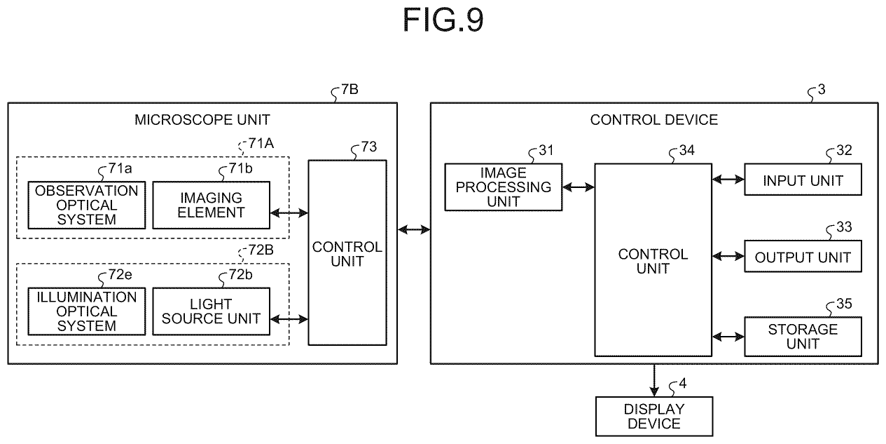

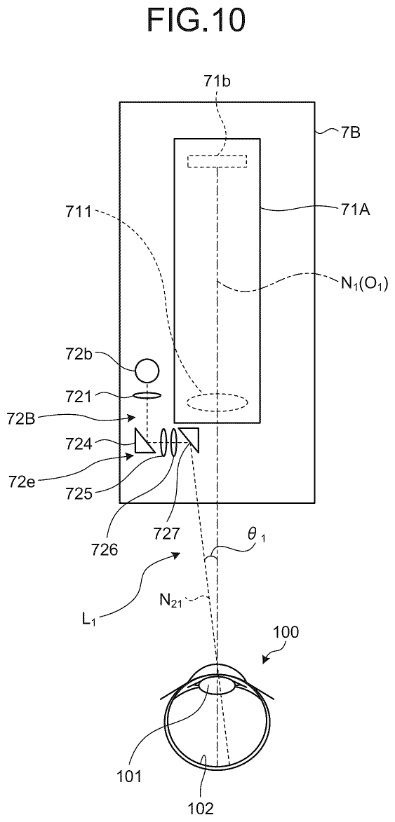

Next, a third modification of the first embodiment will be described with reference to FIGS. 9 to 11. FIG. 9 is a block diagram illustrating a configuration of the surgical microscope system according to the third modification of the first embodiment of the present disclosure. FIG. 10 is a diagram illustrating a configuration of the microscope unit of the surgical microscope system according to the third modification of the first embodiment of the present disclosure. The configuration of the surgical microscope system according to the third modification includes a microscope unit 7B instead of the microscope unit 7 of the surgical microscope system 1 of the first embodiment described above. Since the configuration other than the microscope unit 7B is the same as that of the first embodiment, the description thereof will be omitted. Hereinafter, portions different from those of the first embodiment will be described.

The microscope unit 7B is a cylindrical casing, and includes an imaging unit 71A that enlarges and captures an image of the object to be observed, an illumination unit 72B that irradiates the object to be observed with illumination light, and the control unit 73 that controls the imaging unit 71A and each illumination unit 72B under the control of the control device 3.

The imaging unit 71A captures an image of a subject by taking in observation light. The imaging unit 71A accommodates, in the casing, the observation optical system 71a that includes a plurality of lenses such as an objective lens 713 to form an image of observation light, and the imaging element 71b that takes in the observation light entering through the observation optical system 71a to generate an electric signal. The imaging unit 71A is a monocular imaging unit. The observation optical system 71a and the imaging element 71b are the same as those in the first embodiment, but the description will be given assuming that the observation optical axis N₁ of the imaging unit 71A coincides with a first axis O₁. The position of the observation optical axis N₁ is not limited thereto.

The illumination unit 72B includes an illumination optical system 72e and the light source unit 72b.

The illumination optical system 72e guides the light emitted from the light source unit 72b and emits the guided light to the outside (object to be observed). The illumination optical system 72e includes the light source lens 721, the second mirror 724, the relay lenses 725 and 726, and the third mirror 727. The illumination optical system 72e is configured not to include the half mirror 722, the first mirror 723, the fourth mirror 728, the fifth mirror 729, the sixth mirror 730, the relay lenses 731 and 732, and the seventh mirror 733 with respect to the illumination optical system 72a, and forms one optical path L₁ in which the optical path is not split. In the illumination optical system 72e, the light emitted from the light source unit 72b is guided from the second mirror 724 to the vicinity of the observation optical axis N₁ via the relay lenses 725 and 726, and is guided by the third mirror 727 to travel toward the object to be observed. Specifically, in the illumination optical system 72e, the light emitted from the light source unit 72b is bent by the second mirror 724 via the light source lens 721. The light bent by the second mirror 724 is bent by the third mirror 727 via the relay lenses 725 and 726. The light bent by the third mirror 727 is emitted to the outside as the illumination light.

The illumination optical system 72e forms the optical path L₁ independent of the observation optical system 71a. The optical path L₁ does not intersect or overlap with an optical path from the objective lens 711 to the imaging element 71b in the observation optical system 71a.

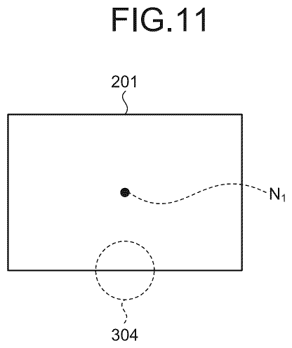

FIG. 11 is a diagram illustrating an angle of view and an illumination position of the microscope unit of the surgical microscope system according to the third modification of the first embodiment of the present disclosure. In the microscope unit 7B, the third mirror 727 emits an illumination light 304 from a position partially overlapping the edge end of the angle of view 201.

An angle θ1 formed by the observation optical axis N₁ of the imaging unit 71A and the optical axis (illumination optical axis N 2) of the illumination light emitted from the third mirror 727 is 2.0 degrees or less, and more preferably 1.5 degrees or less. In addition, the illumination light emitted from the third mirror 727 of the illumination unit 72B intersects with the observation optical axis N₁. For example, when the object to be observed is the eye 100 illustrated in FIG. 10, the illumination light intersects in the eye 100, for example, on the lens 101 or the retina 102.

In the third modification described above, the illumination optical system 72e is configured independently of the observation optical system 71a, and the illumination light is emitted to the outside of the microscope unit 7B without passing through the objective lens 711 via the illumination optical system 72a. According to the third modification, since the illumination light is emitted without passing through the observation optical system 71a, it is possible to suppress flare generated by reflection of the illumination light inside the observation optical system.

### Fourth Modification of First Embodiment

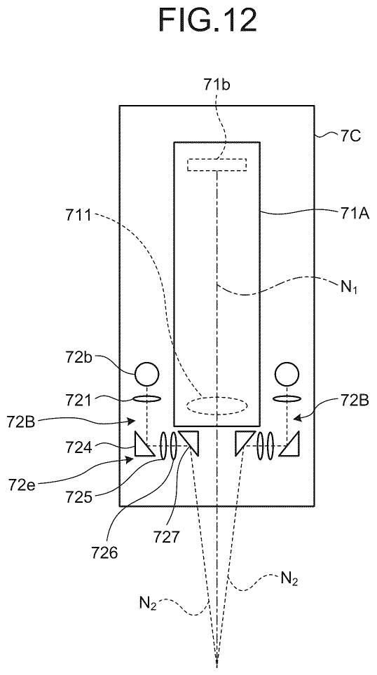

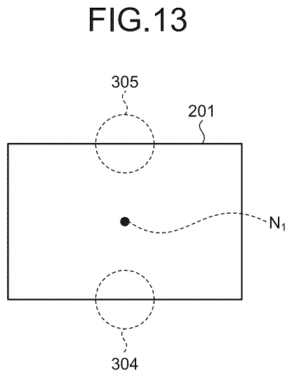

Next, a first modification of the first embodiment will be described with reference to FIGS. 12 and 13. FIG. 12 is a diagram illustrating a configuration of the microscope unit of the surgical microscope system according to the fourth modification of the first embodiment of the present disclosure. The configuration of the surgical microscope system according to the fourth modification includes a microscope unit 7C instead of the microscope unit 7B of the surgical microscope system 1 of the first embodiment described above. Since the configuration other than the microscope unit 7C is the same as that of the first embodiment, the description thereof will be omitted. Hereinafter, portions different from the first embodiment and the third modification will be described.

The microscope unit 7C is a cylindrical casing, and includes the imaging unit 71A that enlarges and captures an image of the object to be observed, two illumination units 72B that irradiate the object to be observed with illumination light, and the control unit 73 (see FIG. 9) that controls the imaging unit 71A and each of the illumination units 72B under the control of the control device 3.

Each of the two illumination units 72B includes the illumination optical system 72e and the light source unit 72b. The illumination units 72B are provided on opposite sides of the observation optical axis N₁ of the imaging unit 71. In other words, the illumination units 72B are arranged on the sides facing each other with the observation optical axis N₁ interposed therebetween.

An angle formed by the observation optical axis N₁ of the imaging unit 71A and the optical axis (illumination optical axis N₂) of the illumination light emitted from the third mirror 727 of each of the illumination units 72B is 2.0 degrees or less. The angles may be the same or different from each other. In addition, it is preferable that the illumination lights emitted from the third mirrors 727 of the illumination units 72B intersect on the observation optical axis N₁. When the object to be observed is an eye, the illumination lights intersect in the eye, for example, on the lens or the retina.

FIG. 13 is a diagram illustrating an angle of view and an illumination position of the microscope unit of the surgical microscope system according to the fourth modification of the first embodiment of the present disclosure. In the microscope unit 7B, the third mirrors 727 emit the illumination lights 304 and 305 from positions partially overlapping the edge end of the angle of view 201. Here, the illumination lights 304 and 305 are located on opposite sides of the observation optical axis N₁. The illumination lights 304 and 305 are positioned, for example, in the vertical direction of the eye that is the object to be observed.

In the fourth modification described above, similarly to the first embodiment, the illumination optical system 72e is configured independently of the observation optical system 71a, and the illumination light is emitted to the outside of the microscope unit 7B through the illumination optical system 72e without passing through the objective lens 711. According to the fourth modification, since the illumination light is emitted without passing through the observation optical system 71a, it is possible to suppress flare generated by reflection of the illumination light inside the observation optical system.

In the fourth modification, the illumination optical system 72e is formed with the observation optical axis N₁ interposed therebetween, and the object to be observed is irradiated with the illumination light from different directions with respect to the observation optical axis N₁. According to the fourth modification, the object to be observed is irradiated with the illumination light of about twice the light amount as compared with the third modification. As a result, high visibility of the object to be observed can be realized.

### Fifth Modification of First Embodiment

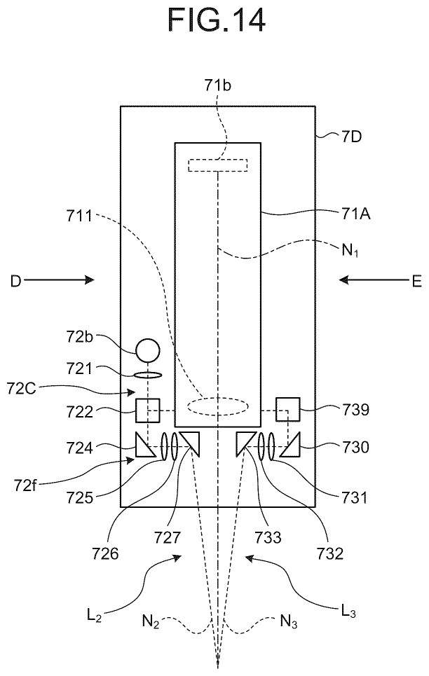

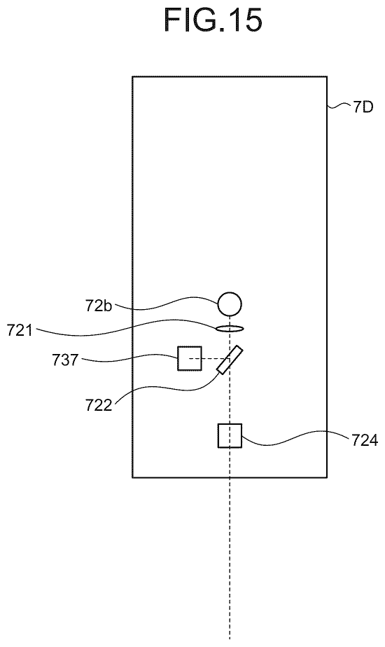

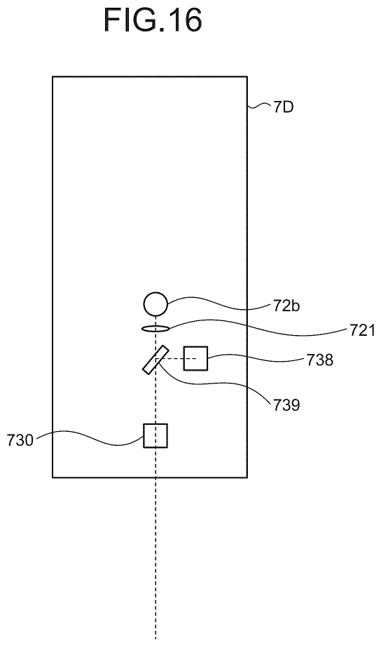

Next, a fifth modification of the first embodiment will be described with reference to FIGS. 14 to 16. FIG. 14 is a diagram illustrating a configuration of the microscope unit of the surgical microscope system according to the fifth modification of the first embodiment of the present disclosure. FIG. 15 is a diagram illustrating a configuration of the microscope unit viewed from a direction of an arrow D in FIG. 14. FIG. 16 is a diagram illustrating a configuration of the microscope unit viewed from a direction of an arrow E illustrated in FIG. 14. The configuration of the surgical microscope system according to the fifth modification includes a microscope unit 7D instead of the microscope unit 7 of the surgical microscope system 1 of the first embodiment described above. Since the configuration other than the microscope unit 7D is the same as that of the first embodiment, the description thereof will be omitted. Hereinafter, portions different from the first embodiment and the third modification will be described.

The microscope unit 7D includes an illumination unit 72C instead of the illumination unit 72B of the microscope unit 7B described above. Since the configuration other than the illumination unit 72C is the same as that of the first embodiment and the third modification, the description thereof will be omitted.

The illumination unit 72C includes an illumination optical system 72f and the light source unit 72b.

The illumination optical system 72f guides light emitted from the light source unit 72b and emits illumination light from the opposite side with the observation optical axis N₁ interposed therebetween. The illumination optical system 72f includes the light source lens 721, the half mirror 722, the second mirror 724, the relay lenses 725 and 726, the third mirror 727, a ninth mirror 737, a tenth mirror 738, an eleventh mirror 739, the sixth mirror 730, the relay lenses 731 and 732, and the seventh mirror 733.

In the illumination optical system 72f, the light is split into two optical paths by the half mirror 722.

In one optical path, light emitted from the light source unit 72b passes through the half mirror 722 via the light source lens 721 and is bent by the second mirror 724. The light bent by the second mirror 724 is bent by the third mirror 727 via the relay lenses 725 and 726. The light bent by the third mirror 727 is emitted to the outside as the illumination light.

In the other optical path, the light emitted from the light source unit 72b is bent by the half mirror 722 via the light source lens 721 and then bent by the ninth mirror 737. The light bent by the ninth mirror 737 is bent by the tenth mirror 738 and the eleventh mirror 739, and passes around the imaging unit 71A. Then, the light bent by the eleventh mirror 739 is bent by the sixth mirror 730, and then bent by the seventh mirror 733 through the relay lenses 731 and 732. The light bent by the seventh mirror 733 is emitted to the outside as the illumination light.

Here, the illumination optical system 72f is provided at a position deviated from the optical path of the observation light entering the imaging unit 71A. In FIG. 14, the third mirror 727 and the seventh mirror 733 are provided at positions that do not disturb the light to be taken in by the objective lens 711. The illumination optical system 72f forms independent optical paths L₂ and L₃ different from the observation optical system 71a. The optical paths L₂ and L₃ do not overlap with the optical path from the objective lens 711 to the imaging element 71b in the observation optical system 71a.

Angles formed by the observation optical axis N₁ of the imaging unit 71A and the optical axes of the illumination light (Illumination optical axes N₂ and N₃) respectively emitted from the third mirror 727 and the seventh mirror 733 is 2.0 degrees or less. The angles formed by the illumination lights and the observation optical axis N₁ may be the same or different from each other. In addition, the illumination lights emitted from the illumination unit 72C preferably intersect on the observation optical axis N₁.

In the fifth modification described above, similarly to the first embodiment, the illumination optical system 72f is configured independently of the observation optical system 71a, and the illumination light is emitted to the outside of the microscope unit 7D without passing through the objective lens 711 via the illumination optical system 72f. According to the fifth modification, since the illumination light is emitted without passing through the observation optical system 71a, it is possible to suppress flare generated by reflection of the illumination light inside the observation optical system.

Still more, in the fifth modification, the illumination optical system 72f irradiates the object to be observed with the illumination light from different directions with respect to the observation optical axis N₁. According to the fifth modification, since the object to be observed is irradiated with the illumination light from the different directions high visibility of the object to be observed can be realized as compared with the first embodiment.

### Sixth Modification of First Embodiment

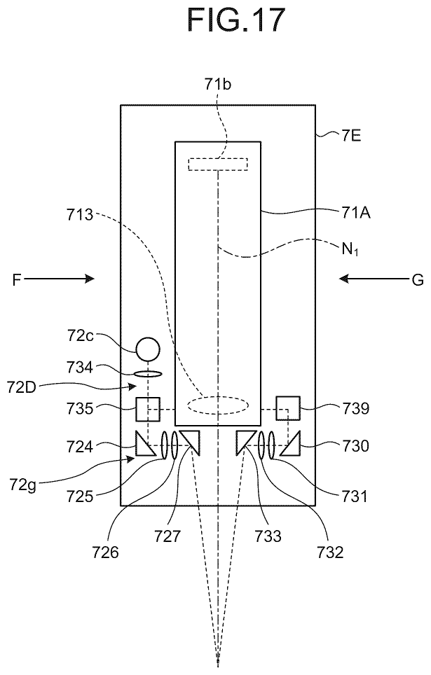

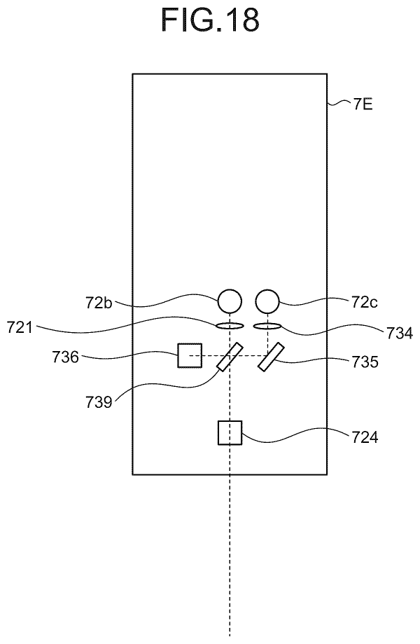

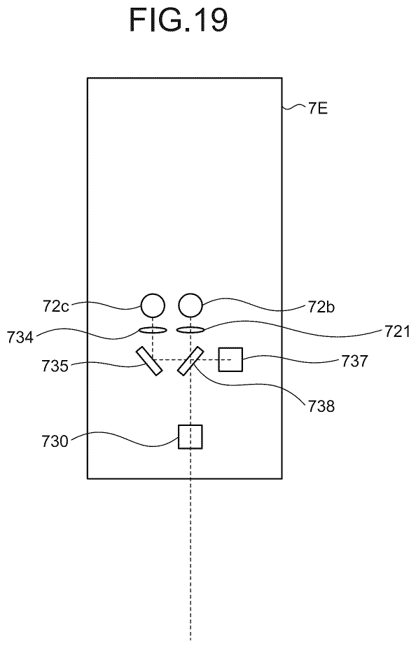

Next, a sixth modification of the first embodiment will be described with reference to FIGS. 17 to 19. FIG. 17 is a diagram illustrating a configuration of the microscope unit of the surgical microscope system according to the sixth modification of the first embodiment of the present disclosure. FIG. 18 is a diagram illustrating a configuration of the microscope unit viewed from a direction of an arrow F in FIG. 17. FIG. 19 is a diagram illustrating a configuration of the microscope unit viewed from a direction of an arrow G in FIG. 17. The configuration of the surgical microscope system according to the sixth modification includes a microscope unit 7E instead of the microscope unit 7 of the surgical microscope system 1 of the first embodiment described above. Since the configuration other than the microscope unit 7E is the same as that of the first embodiment, the description thereof will be omitted. Hereinafter, portions different from the first embodiment and the fifth modification will be described.

The microscope unit 7E includes an illumination unit 72B instead of the illumination unit 72 of the microscope unit 7 described above. The configuration other than illumination unit 72B is the same as that of the first embodiment, and thus the description thereof is omitted.

An illumination unit 72D includes an illumination optical system 72g, the first light source unit 72b, and the second light source unit 72c.

The illumination optical system 72g guides light emitted from the first light source unit 72b or the second light source unit 72c, and emits illumination light from the opposite side with the observation optical axis N₁ interposed therebetween. The illumination optical system 72d includes the light source lens 721, the half mirror 736, the second mirror 724, the relay lenses 725 and 726, the third mirror 727, the ninth mirror 737, the tenth mirror 738, the eleventh mirror 739, the sixth mirror 730, the relay lenses 731 and 732, the seventh mirror 733, the light source lens 734, and the eighth mirror 735.

In the illumination optical system 72g, the light is split into two optical paths by the half mirror 736, similarly to the illumination optical system 72f. Specifically, the light emitted from the first light source unit 72b passes through the light source lens 721, partially passes through the half mirror 736, and the remaining light is bent by the half mirror 736. On the other hand, the light emitted from the second light source unit 72c is bent by the eighth mirror 735 via the light source lens 734, a part of the bent light passes through the half mirror 736, and the remaining light is bent by the half mirror 736. The optical path after passing through the half mirror 736 is similar to that of the illumination optical system 72f.

In the sixth modification described above, similarly to the first embodiment, the illumination optical system 72g is configured independently of the observation optical system 71a, and the illumination light is emitted to the outside of the microscope unit 7D without passing through the objective lens 711 via the illumination optical system 72g. According to the sixth modification, since the illumination light is emitted without passing through the observation optical system 71a, it is possible to suppress flare generated by reflection of the illumination light inside the observation optical system.

Still more, in the sixth modification, the illumination optical system 72g irradiates the object to be observed with the illumination light from a different direction with respect to the observation optical axis N₁. According to the sixth modification, since the object to be observed is irradiated with the illumination light from the different direction, high visibility of the object to be observed can be realized as compared with the first embodiment.

Furthermore, according to the sixth modification, it is possible to selectively switch a wavelength of the light with which the object to be observed is irradiated by switching the driving of the light sources that emit the light in wavelength bands different from each other.

### Second Embodiment

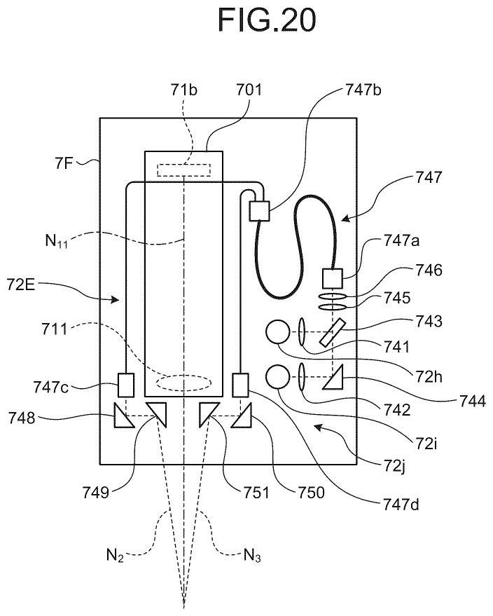

Next, a second embodiment will be described with reference to FIG. 20. FIG. 20 is a diagram illustrating a configuration of a microscope unit of a surgical microscope system according to the second embodiment of the present disclosure. The configuration of the surgical microscope system according to the second embodiment includes a microscope unit 7F instead of the microscope unit 7 of the surgical microscope system 1 of the first embodiment described above. Since the configuration other than the microscope unit 7F is the same as that of the first embodiment, the description thereof will be omitted. Hereinafter, portions different from those of the first embodiment will be described.

The microscope unit 7F includes an illumination unit 72E instead of the illumination unit 72 of the microscope unit 7 described above. Since the configuration other than the illumination unit 72E is the same as that of the first embodiment, the description thereof will be omitted.

The illumination unit 72E includes a first light source unit 72h, a second light source unit 72i, and an illumination optical system 72j.

The first light source unit 72h emits light including light in a wavelength band necessary for observation, such as white light, as illumination light. For example, the white light includes light in all wavelength bands in a visible range.

The second light source unit 72i is light of a wavelength band different from the wavelength band in the visible range. Similarly to the second light source unit 72c, the second light source unit 72i emits light of a wavelength band outside the visible range (e.g., near infrared range) or light of a partial wavelength range of the wavelength band in the visible range (e.g., a part of a green wavelength band, a part of a blue wavelength band, or a wavelength band obtained by a combination thereof) which is used for observation different from the white light.

The illumination optical system 72j guides the light emitted from the first light source unit 72h or the second light source unit 72i and emits the guided light to the outside (object to be observed). The illumination optical system 72j includes light source lenses 741 and 742, a half mirror 743, a first mirror 744, relay lenses 745 and 746, an optical fiber 747, a second mirror 748, a third mirror 749, a fourth mirror 750, a fifth mirror 751, a sixth mirror 752, a seventh mirror 753, an eighth mirror 754, and a ninth mirror 755. The half mirror 743 transmits, for example, half light with respect to a light amount and bends remaining half light. In FIG. 20, the sixth mirror 752 to the ninth mirror 755 are omitted, but have the same functions as the second mirror 748 to the fifth mirror 751.

The optical fiber 747 has an incident end 747a, a splitting unit 474b, and four emission ends (first emission end 747c to fourth emission end 747f, only the first emission end 747c and the second emission end 747d are illustrated in FIG. 20).

In the illumination optical system 72j, the optical fiber 747 splits the light into four optical paths. Specifically, the light emitted from the first light source unit 72h passes through the light source lens 741 and is partially bent by the half mirror 743. On the other hand, the light emitted from the second light source unit 72i is bent by the first mirror 744 through the light source lens 742, and a part of the bent light passes through the half mirror 743. The light entering the relay lenses 745 and 746 via the half mirror 743 enters the incident end 747a of the optical fiber 747. The light entering the optical fiber 747 is split into four optical paths at a splitting unit 747b, and is emitted from the first emission end 747c to the fourth emission end 747f. The light emitted from the first emission end 747c is bent by the second mirror 748 and the third mirror 749 and emitted to the outside. The light emitted from the second emission end 747d is bent by the fourth mirror 750 and the fifth mirror 751 and emitted to the outside. The light emitted from a third emission end 747e is bent by the sixth mirror 752 and the seventh mirror 753 and emitted to the outside. The light emitted from the fourth emission end 747f is bent by the eighth mirror 754 and the ninth mirror 755 and emitted to the outside.

Here, the illumination optical system 72j is provided at a position deviated from the optical path of the observation light entering the imaging unit 71. In FIG. 20, the third mirror 749 and the fifth mirror 751 are provided at positions that do not disturb the light to be taken in by the objective lens 711. In addition, the seventh mirror 753 and the ninth mirror 755 are provided at positions that do not disturb the light to be taken in by the objective lens 712.

Angles formed by the observation optical axis N₁₁ of the imaging unit 71 and optical axes of the illumination light (illumination optical axes N₂ and N₃) respectively emitted from the third mirror 749 and the fifth mirror 751 are 2.0 degrees or less. In addition, angles formed by the observation optical axis N₁₂ of the imaging unit 71 and the optical axes (illumination optical axes N₂ and N₃) of the illumination light respectively emitted from the seventh mirror 753 and the ninth mirror 755 are 2.0 degrees or less. The angles formed by the illumination light and the observation optical axis may be the same or different from each other. In addition, it is preferable that the illumination lights intersect with each other on the observation optical axis.

In the second embodiment described above, the illumination optical system 72j is configured independently of the observation optical systems 71a and 71c, and the illumination light is emitted to the outside of the microscope unit 7F via the illumination optical system 72j without passing through the objective lenses 711 and 712. According to the second embodiment, since the illumination light is emitted without passing through the observation optical systems 71a and 71c, it is possible to suppress flare generated by reflection of the illumination light inside the observation optical system.

In the second embodiment, the illumination optical system 72j irradiates the object to be observed with the illumination light from different directions with respect to the observation optical axes N₁₁ and N₁₂. According to the second embodiment, since the object to be observed is irradiated with the illumination light from different directions from each other, high visibility of the object to be observed can be realized.

Furthermore, according to the second embodiment, it is possible to selectively switch the wavelength of the light with which the object to be observed is irradiated by switching the driving of the light sources that emit the light in the wavelength bands different from each other.

Note that, in the second embodiment, either the first light source unit 72h or the second light source unit 72i may be included. When only the first light source unit 72h is provided, a mirror is provided instead of the half mirror 743, and the second light source unit 72i, the light source lens 742, and the first mirror 744 are not provided. When only the second light source unit 72i is provided, the first light source unit 72h, the light source lens 741, and the half mirror 743 are not provided.

### Third Embodiment

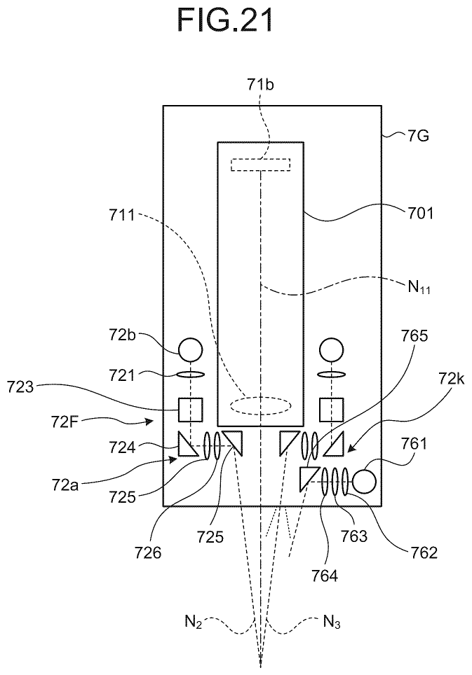

Next, a third embodiment will be described with reference to FIG. 21. FIG. 21 is a diagram illustrating a configuration of a microscope unit of a surgical microscope system according to the third embodiment of the present disclosure. The configuration of the surgical microscope system according to the third embodiment includes a microscope unit 7G instead of the microscope unit 7 of the surgical microscope system 1 of the first embodiment described above. Since the configuration other than the microscope unit 7G is the same as that of the first embodiment, the description thereof will be omitted. Hereinafter, portions different from those of the first embodiment will be described.

The microscope unit 7G includes, in a cylindrical casing, the imaging unit 71 that enlarges and captures an image of the object to be observed, an illumination unit 72F that irradiates an object to be observed with illumination light, and the control unit 73 (see FIG. 2) that controls the imaging unit 71 and the illumination unit 72F under the control of the control device 3.

The illumination unit 72F includes the illumination optical system 72a, the light source unit 72b, and a wide-area illumination unit 72k. Since the configuration other than the wide-area illumination unit 72k is the same as that of the first embodiment, the description thereof will be omitted.

The wide-area illumination unit 72k includes a light source unit 761, a relay lenses 762 to 764, and a mirror 765.

The light source unit 761 emits light including light in a wavelength band necessary for observation, such as white light, as illumination light. For example, the white light includes light in all wavelength bands in a visible range.

The light emitted from the light source unit 761 passes through the relay lenses 762 to 764, is bent by the mirror 765, and is emitted to the outside. The illumination light emitted from the wide-area illumination unit 72k illuminates an object to be observed over a wide area as compared with the illumination optical system 72a. For example, the wide-area illumination unit 72k illuminates an area around the eye including the eye that is a surgery target to be observed.

In the third embodiment described above, similarly to the first embodiment, the illumination optical system 72a is configured independently of the observation optical system 71a, and the illumination light is emitted to the outside of the microscope unit 7G through the illumination optical system 72a without passing through the objective lens 711. According to the third embodiment, since the illumination light is emitted without passing through the observation optical system 71a, it is possible to suppress flare caused by reflection of the illumination light inside the observation optical system.

Still more, in the third embodiment, since the area around the surgery target is illuminated in a wide range by the wide-area illumination unit 72k provided separately from the illumination optical system 72a, a work area of the operator or the like can be further brightened.

Furthermore, in the third embodiment, since the wide-area illumination unit 72k obliquely illuminates the object to be observed, the image of the object to be observed is shaded, and thus an image having a stereoscopic effect can be obtained.

### Fourth Embodiment

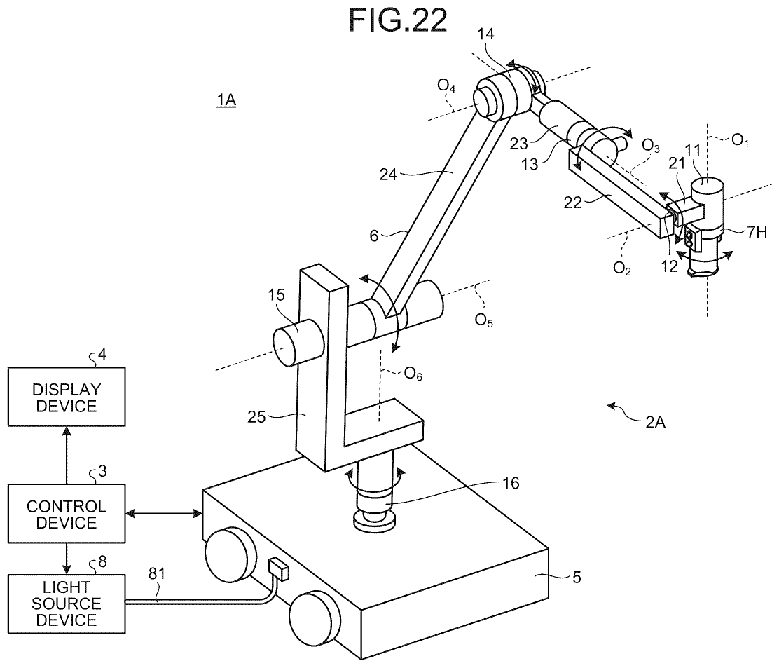

Next, a fourth embodiment will be described with reference to FIG. 22. FIG. 22 is a diagram illustrating a configuration of a surgical microscope system according to the fourth embodiment of the present disclosure. A surgical microscope system 1A includes the control device 3 and the display device 4 described above, a microscope device 2A, and a light source device 8 that supplies illumination light to the microscope device 2A. Since the configuration other than the microscope device 2A and the light source device 8 is the same as that of the first embodiment, the description thereof will be omitted. Hereinafter, portions different from those of the first embodiment will be described.

The microscope device 2A includes a microscope unit 7H instead of the microscope unit 7 of the microscope device 2 described above. Since the configuration other than the microscope unit 7H is the same as that of the first embodiment, the description thereof will be omitted. Hereinafter, portions different from those of the first embodiment will be described.

The microscope unit 7H includes the above-described illumination optical system 72a. In other words, the microscope unit 7H does not include the light source unit 72b with respect to the configuration of the microscope unit 7.

In the surgical microscope system 1A, the microscope device 2A includes, in addition to a transmission cable including a signal line for performing signal transmission between the control device 3 and the microscope unit 7H, a light guide cable for guiding the illumination light from the light source device 8 to the microscope unit 7.

The light source device 8 includes a light source, and controls emission of light under the control of the control device 3. The light source device 8 is connected to the microscope device 2 via a light source cable 81. An optical fiber is inserted into the light source cable 81.

In the microscope device 2A, an optical cable is provided to be connected to the light source cable 81 and guides light to the microscope unit 7H via each arm and each joint. In the microscope unit 7H, the light guided by the optical cable enters the illumination optical system 72a. The light entering the illumination optical system 72a is emitted to the outside in the same manner as in the above-described first embodiment.

In the fourth embodiment described above, similarly to the first embodiment, the illumination optical system 72a is configured independently of the observation optical systems 71a and 71c, and the illumination light is emitted to the outside of the microscope unit 7H without passing through the objective lenses 711 and 712 via the illumination optical system 72a. According to the fourth embodiment, since the illumination light is emitted without passing through the observation optical system 71a, it is possible to suppress flare caused by reflection of the illumination light inside the observation optical system.

Still more, in the fourth embodiment, the light source device 8 having the light source is provided separate from the microscope unit 7H, and light emitted from the light source device 8 is guided to the microscope unit 7H. According to the fourth embodiment, the light source can be replaced without replacing the microscope unit 7H. In other words, in the fourth embodiment, the wavelength band of the light to be illuminated can be changed without decomposing the microscope unit 7H.

### Other Embodiments

Various inventions can be formed by appropriately combining a plurality of components disclosed in the surgical microscope system according to the embodiments of the present disclosure described above. For example, some components may be deleted from all the components described in the surgical microscope system according to the embodiments of the present disclosure described above. Furthermore, the components described in the surgical microscope system according to the first to fourth embodiments and the modifications of the present disclosure described above may be appropriately combined.

Furthermore, in the surgical microscope system according to the embodiments of the present disclosure, the above-described “unit” can be replaced with “means”, “circuit”, or the like. For example, the control unit can be replaced with a control means or a control circuit.

In addition, the program to be executed by the surgical microscope system according to the embodiments of the present disclosure is provided by being recorded in a computer-readable recording medium such as a CD-ROM, a flexible disk (FD), a CD-R, a digital versatile disk (DVD), a USB medium, or a flash memory as file data in an installable format or an executable format.

Furthermore, the program executed by the surgical microscope system according to the embodiments of the present disclosure may be stored in a computer connected to a network such as the Internet and provided by being downloaded via the network.

Although some of the embodiments of the present disclosure have been described in detail with reference to the drawings, these are merely examples, and the present disclosure can be implemented in other forms subjected to various modifications and improvements based on the knowledge of those skilled in the art, including the aspects described in the present disclosure.

Note that the present technology can also have the following configurations.

(1)

A surgical microscope system including:
an imaging unit including
   an observation optical system configured using a plurality of optical members including an objective lens, and
   an imaging element configured to capture an image of a subject formed by the observation optical system; and
an illumination unit including
   a light source unit configured to emit light, and
   an illumination optical system configured to guide the light emitted by the light source unit, wherein
the illumination optical system is configured to form an illumination optical path that is independent of an observation optical path formed by the observation optical system, the illumination optical path intersecting with the observation optical path on a side of the subject with respect to the objective lens.
   (2)

The surgical microscope system according to (1), wherein the illumination optical system includes an optical splitting unit configured to split the light emitted by the light source unit.

(3)

The surgical microscope system according to (2), wherein the optical splitting unit is a beam splitter.

(4)

The surgical microscope system according to (2), wherein the optical splitting unit is an optical fiber.

(5) The surgical microscope system according to any one of (1) to (4), wherein the illumination optical system is configured to emit illumination light from opposite sides of an observation optical axis of the imaging unit with respect to an angle of view of the imaging unit.
(6)

The surgical microscope system according to any one of (1) to (4), wherein
the imaging unit includes first and second imaging units configured to capture images having parallax, each of the first and second imaging units including the observation optical system and the imaging element, and
the illumination optical system is configured to emit illumination light from opposite sides of an observation optical axis of each of the first and second imaging units with respect to an angle of view of each of the first and second imaging units.
   (7)

The surgical microscope system according to any one of (1) to (6), wherein an angle formed by an illumination optical axis of light emitted toward the subject and an observation optical axis of the imaging unit is not greater than two degrees in the illumination optical system.

(8)

The surgical microscope system according to any one of (1) to (6), wherein the illumination unit includes:
a first light source unit configured to emit light in a first wavelength band, and
a second light source unit configured to emit light in a wavelength band different from the first wavelength band, and
the illumination unit is configured to switch emission of light by the first light source unit and the second light source unit.
   (9)

The surgical microscope system according to (8), wherein
the first light source unit is configured to emit white light in a wavelength band in a visible range, and
the second light source unit is configured to emit near-infrared light in a wavelength band having a center wavelength of 850 nm.
   (10)

The surgical microscope system according to any one of (1) to (9), further including:
a holding unit configured to hold the imaging unit and the illumination optical system;
a support unit configured to support the holding unit;
a control device configured to control the imaging unit and the illumination unit; and
a display device configured to display an image captured by the imaging unit.
   (11)

The surgical microscope system according to (10), wherein the light source unit is provided in the holding unit.

(12)

The surgical microscope system according to (10), wherein the light source unit is configured to supply light to the illumination optical system via the support unit.

### INDUSTRIAL APPLICABILITY

As described above, the surgical microscope system according to the present invention is effective for suppressing flare caused by reflection of illumination light in the observation optical system.

### REFERENCE SIGNS LIST

1, 1A SURGICAL MICROSCOPE SYSTEM
2, 2A MICROSCOPE DEVICE
3 CONTROL DEVICE
4 DISPLAY DEVICE
5 BASE UNIT
6 SUPPORT UNIT
7, 7A to 7H MICROSCOPE UNIT
8 LIGHT SOURCE DEVICE
11 FIRST JOINT
12 SECOND JOINT
13 THIRD JOINT
14 FOURTH JOINT
15 FIFTH JOINT
16 SIXTH JOINT
21 FIRST ARM
22 SECOND ARM
23 THIRD ARM
24 FOURTH ARM
25 FIFTH ARM
31 IMAGE PROCESSING UNIT
32 INPUT UNIT
33 OUTPUT UNIT
34, 73 CONTROL UNIT
35 STORAGE UNIT
71, 71A IMAGING UNIT
71a, 71c OBSERVATION OPTICAL SYSTEM
71b, 71d IMAGING ELEMENT
72, 72A to 72F ILLUMINATION UNIT
72a, 72d, 72e, 72f, 72g, 72j ILLUMINATION OPTICAL SYSTEM
72b, 761 LIGHT SOURCE UNIT (FIRST LIGHT SOURCE UNIT)
72h FIRST LIGHT SOURCE UNIT
72c, 72i SECOND LIGHT SOURCE UNIT
72k WIDE-AREA ILLUMINATION UNIT
81 LIGHT SOURCE CABLE
711, 712 OBJECTIVE LENS
721, 734, 741, 742 LIGHT SOURCE LENS
722, 736, 743 HALF MIRROR
723, 744 FIRST MIRROR
724, 748 SECOND MIRROR
725, 726, 731, 732, 745, 746, 762 to 764 RELAY LENS
727, 749 THIRD MIRROR
728, 750 FOURTH MIRROR
729, 751 FIFTH MIRROR
730, 752 SIXTH MIRROR
733, 753 SEVENTH MIRROR
735, 754 EIGHTH MIRROR
737, 755 NINTH MIRROR
738 TENTH MIRROR
739 ELEVENTH MIRROR
747 OPTICAL FIBER
765 MIRROR

1. A surgical microscope system comprising:
an imaging unit including
an observation optical system configured using a plurality of optical members including an objective lens, and
an imaging element configured to capture an image of a subject formed by the observation optical system; and
an illumination unit including
a light source unit configured to emit light, and
an illumination optical system configured to guide the light emitted by the light source unit, wherein
the illumination optical system is configured to form an illumination optical path that is independent of an observation optical path formed by the observation optical system, the illumination optical path intersecting with the observation optical path on a side of the subject with respect to the objective lens.

2. The surgical microscope system according to claim 1, wherein the illumination optical system includes an optical splitting unit configured to split the light emitted by the light source unit.

3. The surgical microscope system according to claim 2, wherein the optical splitting unit is a beam splitter.

4. The surgical microscope system according to claim 2, wherein the optical splitting unit is an optical fiber.

5. The surgical microscope system according to claim 1, wherein the illumination optical system is configured to emit illumination light from opposite sides of an observation optical axis of the imaging unit with respect to an angle of view of the imaging unit.

6. The surgical microscope system according to claim 1, wherein
the imaging unit includes first and second imaging units configured to capture images having parallax, each of the first and second imaging units including the observation optical system and the imaging element, and
the illumination optical system is configured to emit illumination light from opposite sides of an observation optical axis of each of the first and second imaging units with respect to an angle of view of each of the first and second imaging units.

7. The surgical microscope system according to claim 1, wherein an angle formed by an illumination optical axis of light emitted toward the subject and an observation optical axis of the imaging unit is not greater than two degrees in the illumination optical system.

8. The surgical microscope system according to claim 1, wherein the illumination unit includes:
a first light source unit configured to emit light in a first wavelength band, and
a second light source unit configured to emit light in a wavelength band different from the first wavelength band, and
the illumination unit is configured to switch emission of light by the first light source unit and the second light source unit.

9. The surgical microscope system according to claim 8, wherein
the first light source unit is configured to emit white light in a wavelength band in a visible range, and
the second light source unit is configured to emit near-infrared light in a wavelength band having a center wavelength of 850 nm.

10. The surgical microscope system according to claim 1, further comprising:
a holding unit configured to hold the imaging unit and the illumination optical system;
a support unit configured to support the holding unit;
a control device configured to control the imaging unit and the illumination unit; and
a display device configured to display an image captured by the imaging unit.

11. The surgical microscope system according to claim 10, wherein the light source unit is provided in the holding unit.

12. The surgical microscope system according to claim 10, wherein the light source unit is configured to supply light to the illumination optical system via the support unit.
